(19) United States
(12) Patent Application Publication
(10) Pub. No.: US 2024/0068873 A1
(43) Pub. Date: Feb. 29, 2024

(54) COLOR CODE AUTHENTICATION OF A DRUG CARTRIDGE USING SINGLE-PIXEL COLOR IMAGING SENSOR

(71) Applicant: TWENTY TWENTY THERAPEUTICS LLC, South San Francisco, CA (US)

(72) Inventor: Timothy Stowe, Alameda, CA (US)

(21) Appl. No.: 18/237,830

(22) Filed: Aug. 24, 2023

Related U.S. Application Data
(60) Provisional application No. 63/400,802, filed on Aug. 25, 2022.

(51) Int. Cl. G01J 3/46; G01J 3/50; G06K 7/14

(52) U.S. Cl. CPC G01J 3/463; G01J 3/501; G06K 7/1417

(57) ABSTRACT

A cartridge storing a therapeutic agent can be authenticated before the therapeutic agent is applied to a user using an inexpensive process involving cloud storage and color detection. An applicator for delivering the therapeutic agent can include a space for receiving a cartridge that can be selected from a group of cartridges, a color sensor configured to detect the color of a color-coded identifying mark on the selected cartridge, and a processor that can receive profile information from a cloud service, including color information of what the color-code identifying mark should be, and match the color of the color-coded identifying mark on the cartridge with the color information. The processor can control if the applicator allows delivery of the therapeutic agent based on the authentication. The profile information can be uploaded by scanning a QR code on the cartridge and uploading associated data to the cloud service.

### CROSS-REFERENCE TO RELATED APPLICATIONS

This application claims the benefit of U.S. Provisional Application Ser. No. 63/400,802, filed 25 Aug. 2022, entitled “COLOR CODE AUTHENTICATION OF A DRUG CARTRIDGE USING SINGLE-PIXEL COLOR IMAGING SENSOR”. The entirety of this application is incorporated by reference for all purposes.

### TECHNICAL FIELD

The present disclosure relates generally to drug cartridge authentication, and more specifically, to detecting a color code on a drug cartridge using a single-pixel color imaging sensor to authenticate the drug cartridge before the drug can be applied to a patient via an applicator.

### BACKGROUND

Many patients suffering from ophthalmic and/or respiratory diseases may need to take one or more medications throughout a day using an applicator or similar-looking applicators (e.g., inhaler, eye drop mister, etc.). For example, patients with chronic obstructive pulmonary disease (COPD) often take two or more inhaler medications per day and glaucoma patients with vision loss often take two or more different eye medications per day. Such patients can mix up which medication(s) they need to take at certain times, as well as which medication(s) they have already taken, especially when using only a single applicator for multiple medicines. A way to track which medications have been properly dosed by a patient via the same applicator within a given time period (e.g., a prescription-indicated time, a week, a day, a morning/evening, etc.) is needed. Verifying that a given patient is not using the wrong medication or a knockoff version of the medication (e.g., that may not include the appropriate chemical compounds) and stopping the user from applying the wrong or knockoff medication is also important. Patients also have a habit of using older or expired medications (e.g., that have lower efficacy) or not renewing a prescription when a cartridge volume is low, so there is a need to track the age of a cartridge and to alert a patients or a patient's caregiver when the cartridge is expiring and/or the volume is low. Cost is often a significant barrier to monitoring patient compliance therefore a low-cost method for solving all of these issues (tracking application timing, verifying the authenticity, amount, and expiration of the medication, etc.) is needed.

### SUMMARY

The present disclosure describes a low-cost authentication method for authenticating a drug cartridge that supplies a therapeutic agent for application to a patient. The authentication method utilizes a single-pixel color imaging sensor to detect a color marked on the drug cartridge in a color-coded identifying mark and authenticates the drug cartridge based on the color and information associated with the drug cartridge in a cloud service, such as cloud-stored database. The entry in the cloud-stored database can be created based on information stored in a bar-code printed on the drug cartridge (like a QR-code) or associated cartridge product packaging. Accordingly, the cartridge can be authenticated before a therapeutic agent within the cartridge can be applied via an applicator. The database also can include or be updated with various information about cartridges and therapeutic agents applied to a given user so that the given user's history with the therapeutic agents can be tracked.

In an aspect, the present disclosure includes a system for applying an authenticated therapeutic agent to a user, where the system includes at least one cartridge holding a therapeutic agent and an applicator for delivering the therapeutic agent inside the cartridge. Multiple cartridges can be used with a single applicator. Each cartridge can be distinguished from others by a unique color-coded identifying mark on an exterior of the cartridge and is configured to hold the therapeutic agent. The color-coded identifying mark is unique to the therapeutic agent. Furthermore, the color-code is associated with the bar-code (QR code) information previously established in the cloud and therefore the user is not required to rescan and re-verify the printed bar code with each and every use. The cartridge registration event happens just once at the first original usage of a new cartridge and thereafter, the cartridge use is detected and verified based upon the automatic detection within the applicator of the cartridge's color-code. The applicator is configured to deliver the therapeutic agent from the cartridge to the user. The applicator includes a cartridge receiving space configured to hold a single cartridge selected from the group of cartridges being used by a patient. The applicator also includes a color sensor configured to detect a color of the color-coded identifying mark on the cartridge when the cartridge is in the cartridge receiving space. The applicator further includes at least a processor in communication with a cloud service and configured to receive the color of the color-coded identifying mark on the cartridge from the color sensor; receive profile information from the cloud service, wherein the profile information comprises stored information associated with the at least one cartridge, wherein the stored information comprises at least color information of the color-coded identifying marks of the at least one cartridge; and match the color of the color-coded identifying mark to the color information, wherein: if the color matches, allow the applicator to apply the therapeutic agent, and if the color does not match, stop the applicator from applying the therapeutic agent

In another aspect, the present disclosure includes an applicator device configured to deliver a therapeutic agent to the user. The applicator includes a cartridge receiving space configured to hold a cartridge having a color-coded identifying mark on an exterior of the cartridge, wherein the cartridge is configured to store the therapeutic agent delivered by the applicator. The applicator also includes a color sensor configured to detect a color of the color-coded identifying mark on the exterior of the cartridge, wherein the color-coded identifying mark is unique for the therapeutic agent. The applicator also includes at least a processor in communication with a cloud service and configured to: receive the color of the color-coded identifying mark on the cartridge from the color sensor; receive profile information from the cloud service, wherein the profile information comprises stored information associated with the cartridge, wherein the stored information comprises at least color information of the color-coded identifying marks of at least one cartridge associated with the user; and match the color of the color-coded identifying mark to the color information, wherein: if the color matches, allow the applicator to apply the therapeutic agent, and if the color does not match, stop the applicator from applying the therapeutic agent.

In another aspect, the present disclosure includes a method for authenticating a cartridge holding a therapeutic agent. The method includes scanning, via a system comprising a processor, a bar-code (e.g., a QR code) on an exterior of a cartridge storing a therapeutic agent or the associated packaging the cartridge comes inside when it is purchased. This bar-code is associated with data related to at least color information of a color-coded identifying mark associated with the therapeutic agent and dosage information related to the therapeutic agent for a user of the therapeutic agent and the system; linking, via the system, the data with a profile of the user on a cloud service; detecting, via a color-sensor of the system, a color of a color-coded identifying mark on the cartridge; receiving, by the system, profile information from the cloud service, wherein the profile information comprises stored information associated with the at least one cartridge, wherein the stored information comprises at least color information of the color-coded identifying marks of the at least one cartridge; and matching, by the system, the color of the color-coded identifying mark to the color information, wherein: if the color matches, allow the applicator to apply the therapeutic agent, and if the color does not match, stop the applicator from applying the therapeutic agent. After the initial cartridge cloud registration verifying the color-code, if the calculated expiration date is exceeded, the applicator will also disable applying the therapeutic agent from that cartridge.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 shows a diagram of a system that can authenticate at least one drug cartridge containing a therapeutic agent that can be delivered to a patient;

FIG. 2 shows a diagram of the system of FIG. 1 authenticating a drug cartridge;

FIG. 3 shows a diagram of the system of FIG. 1 entering information related to a drug cartridge into a profile database on a cloud service;

FIG. 4 shows a diagram of an example of how the color sensor of FIG. 1 can work;

FIG. 5 shows a process flow diagram illustrating a method for authenticating a cartridge containing a therapeutic agent;

FIG. 6 shows an example bottle with a QR code printed on packaging and a color-coded identifying mark printed on the bottle; and

FIG. 7 shows an example of how at least a portion of FIG. 1 may be used in practice.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “cartridge”, also referred to as “drug cartridge”, refers to a portable and self-contained storehouse for a therapeutic agent. A portion of the drug cartridge may be open or openable for release of the therapeutic agent. The opening may be covered to prevent release of the therapeutic agent. In some instances, the covering can facilitate release of the therapeutic agent from the reservoir when triggered by an applicator device.

As used herein, the term “therapeutic agent”, also referred to as “medication” or “drug”, refers to one or more substance (e.g., liquid, solid, and/or gas) related to the treatment, symptom relief, and/or palliative care of a disease, injury, or other malady. Examples of the therapeutic agent can include one or more pharmaceuticals, saline, or the like.

As used herein, the term “applicator”, also referred to as “applicator device”, refers to any device with which a therapeutic agent can be applied and/or delivered to a patient as a liquid, solid, or gas from a cartridge. Non-limiting examples of an applicator include a mister, an eye drop applicator, an inhaler, and the like.

As used herein, the terms “user”, “subject”, and “patient” can be used interchangeably and refer to any warm-blooded organism including, but not limited to, a human being, a pig, a rat, a mouse, a dog, a cat, a goat, a sheep, a horse, a monkey, an ape, a rabbit, a cow, etc.

### II. Overview

Patients, including those needing to use eye drop applicators, inhalers, and the like, may take or be prescribed to take one or more medications over the same time periods (often having to take two or more different medications within a single day). It would be advantageous if these patients could use a common device to deliver these different medications. However, patients often struggle with accurately maintaining medication dosage schedules and/or properly ordering and using in-date medications. Drug cartridges often can look alike, and patients may forget, or not check, what cartridge is inside an applicator at a given time when they use a single applicator for multiple medications. Patients may also use medications less often then prescribed or in smaller doses for cost saving reasons. Additionally, patients may not know a cartridge is nearing empty or its expiration date or may continue to use a cartridge until it is completely empty, regardless of use by date, for additional cost saving reasons. Indeed, cost is often a significant barrier to patient compliance and a cause for problems. Such errors and problems can be prevented via drug authentication. Accordingly, a low-cost method for drug authentication is needed to ease the patients' struggles and would be a welcome addition to doctors' compliance monitoring to ensure accurate use of prescribed therapeutic agents.

As described herein, the present disclosure relates to such a low-cost method that detects a color code on the drug cartridge using a single-pixel color imaging sensor to authenticate the drug cartridge before the drug can be applied to a patient via an applicator. Information about the cartridge, and the therapeutic agent held inside the cartridge, can be found from a bar-code (e.g., a QR-code) on the cartridge and stored in a profile database on a cloud service, which can be used to authenticate the cartridge the first time it is used and linked to the measured color-code for all subsequent uses in the applicator. The present disclosure can also relate to tracking various information about the cartridges and the therapeutic agents applied to a given user, such as times and quantities of applications, amount of therapeutic agent remaining in a given cartridge, age of a cartridge, etc.

### III. Systems

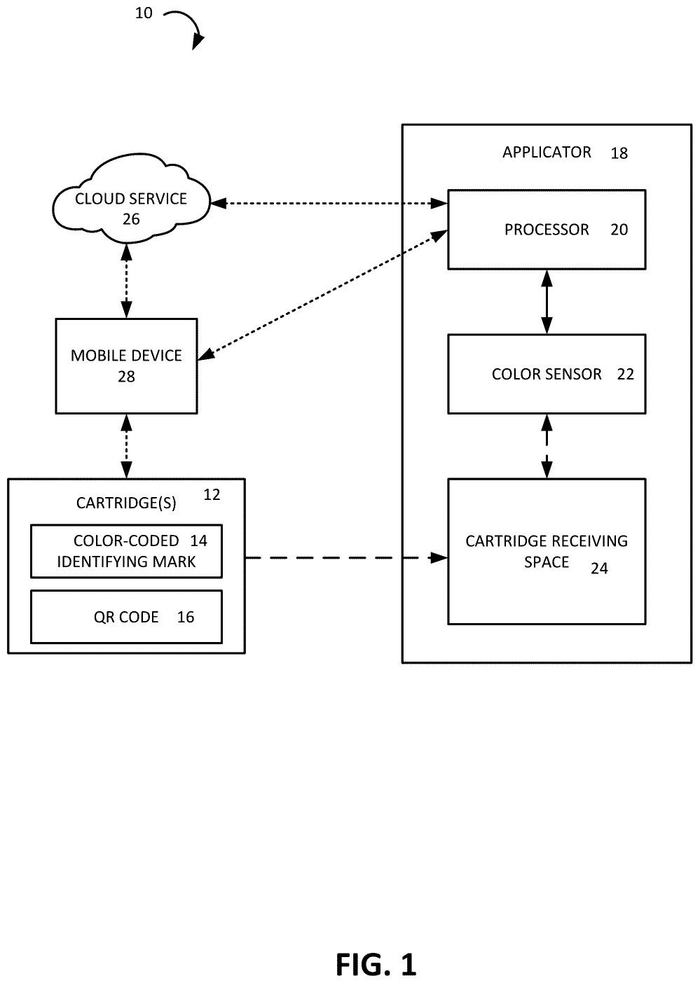

One aspect of the present disclosure includes a system 10 (FIG. 1) that can authenticate and then apply a therapeutic agent in a cartridge (e.g., one or more cartridge(s) 12, example applies to all uses of the word “cartridge”) to a patient using a cloud-based authentication approach and a machine readable color code scheme. Each cartridge (1) can be configured to hold a therapeutic agent for delivery to a patient and (2) can have a color-coded identifying mark 14 directly on the cartridge (also referred to as a color code) and a bar-code (represented by QR code 16 throughout, but this will be understood to be just an example) either also on the exterior of the cartridge or on its associated packaging when purchased (e.g., printed on a label attached thereto, etc.). The color-coded identifying mark 14 can be unique to the therapeutic agent held in each of the cartridge(s) 12. The color code can identify the therapeutic agent based on a color code scheme linked to the QR code. It should be noted that the color code scheme need not conform to an international standard used for people to distinguish the type of ophthalmic therapeutic agent within the cartridge, but the color code can also conform to the international standard. The QR code 16 can be unique to the therapeutic agent as well and may also be unique to the patient and/or a given prescription. The QR code 16 can be associated with data that includes at least the color information of the color-coded identifying mark associated with the therapeutic agent of the at least one cartridge and dosage information related to the therapeutic agent for the user.

The system 10 can authenticate the cartridge and/or the therapeutic agent being held by the cartridge before delivery of the therapeutic agent to the patient. The system 10 can also track information about the at least one cartridge and the patient's uses of the at least one cartridge. As such, the system 10 can provide a way to track which medications have been delivered to the patient via an applicator 18 within a given time period (e.g., a prescription-indicated time, a week, a day, a morning/evening, etc.), verify that a given patient is not using the wrong medication or a knockoff version of the medication (e.g., that may not include the appropriate chemical compounds), stop the user from applying the wrong or knockoff medication, track the age of the cartridge, alert a patients or a patient's caregiver when the cartridge is expiring and/or the volume is low, or the like, all at a low cost. As an example, the tracking can use a cloud service 26 populated using a QR code 16 on the cartridge 12 and accessed using a color code 14 on the cartridge. It should be noted that for a given use of the system 10 a single cartridge can be selected and inserted into the applicator 18. The color code 14 can lead to a low cost approach with zero cost impact on the cartridge as cartridges are already traditionally labeled.

The system 10 can include an applicator 18 that can at least one of detect a presence of the cartridge, detect the color code of the cartridge, and communicate with a cloud service 26 to receive information linked to the cartridge and the applicator 18 (e.g., and stored in one or more databases) through the color code. The applicator 18 can be any device that can be used to deliver the therapeutic agent from a cartridge to at least a portion of a patient's body. For example, the applicator 18 can be an eye drop applicator, an inhaler, or the like. The therapeutic agent can be used for treating ophthalmic conditions, respiratory conditions (e.g., COPD, asthma, or the like), or other conditions/diseases. As an example, the applicator can be used for application of a plurality of different eye drops to treat a plurality of maladies of the eye. Notably, the applicator 18 is not required to detect the cartridge during installation of the cartridge into the applicator 18, so the applicator 18 can remain in a sleep state while different cartridges are being installed.

The applicator 18 can deliver the therapeutic agent from the cartridge to the user. The delivery mechanism of the applicator 18 can depend on the uses of the applicator and types of medications it is meant to dispense and/or deliver (e.g., medications for ocular use, oral use, topical use, etc.). The applicator 18 can include a cartridge receiving space 24 that can hold a cartridge (e.g., the selected cartridge from the group of cartridge(s) 12). The cartridge receiving space 24 can be shielded from external lighting when closed (e.g., during use of the applicator 18 to deliver the therapeutic agent, not when the cartridges are being taken in and out). The applicator 18 can also include a color sensor 22 that can detect a color of the color-coded identifying mark 14 on the cartridge when the cartridge is positioned in the cartridge receiving space 24. The color sensor 22 can detect the color of the color-code identifying mark 14 in a single pixel and is further described below. It should be understood that the single pixel is merely an example that can be extended to two or more pixels such that the number of machine readable combinations can be extended by a factor of 2^{N}, where N is a positive integer. As an example, the color sensor 22 can be a small low cost surface mount optical detector with multiple color filters, such as a TCS34725 available from AMS OSRAM. In particular, color filters with an infrared blocking filter can stabilize color measurements against background temperature changes.

The applicator 18 can also include at least a processor 20 in communication (e.g., wireless, WIFI, Bluetooth®, etc.) with a cloud service 26. In some instances, the communication can be direct between the processor 20 and the cloud service 26. In other instances, the processor 20 can communicate with the cloud service 26 indirectly through the use of a paired mobile device 28 that has an appropriate software application loaded thereon. The applicator 18 may also include a non-transitory memory, a transceiver, a display, or a speaker (not shown). For example, the processor 20 may also be in communication (wired or wireless) with the non-transitory memory that stores instructions for the processor to execute. The processor 20 can execute instructions that include, but are not limited to, receive data, authenticate a cartridge, track information about the cartridge and/or the therapeutic agent.

As stated above, the system 10 can also include a mobile device 28 that can also be in communication with the cloud service 26 and/or the processor 20 (illustrated as broken lines because the communication can be either and/or both). Similarly, the processor 20 can communicate with the cloud services 26 and/or the mobile device 28 (illustrated as broken lines because the communication can be either and/or both).

The mobile device can scan the QR code 16 on the cartridge and communicate with the cloud service 26 to associate the cartridge with the applicator 18 and record the correct color code for that cartridge. The mobile device 28 can also upload additional information about the cartridge based on the QR code, like data associated with the prescription, data associated with expiration of the therapeutic agent, or the like. The mobile device 28 can be a cell phone, a tablet, a laptop, or the like.

The mobile device 28 can be in communication with at least the cloud service 26. The mobile device 28 can be used to scan the QR code 16 on the cartridge(s) 12 to access the data associated with the QR code. The mobile device 28 can upload at least a portion of the data to the cloud service 28 (and, optionally, to an internal memory of the mobile device) and link the at least the portion of the data to the applicator 18 on the cloud service. The mobile device 28 can be, for example, a smartphone, a tablet, a handheld scanner, or the like that includes an image sensor, some form of processor and memory (e.g., at least partially embedded in hardware), and a transceiver. The mobile device 28 may be used to create a profile of a user of the applicator 18 that is stored on a database in the cloud service 26 where the data associated with the QR code is stored. The mobile device 28 can be used to scan a QR code 16 of each cartridge that a patient has as the patient obtains and/or begins to use a cartridge.

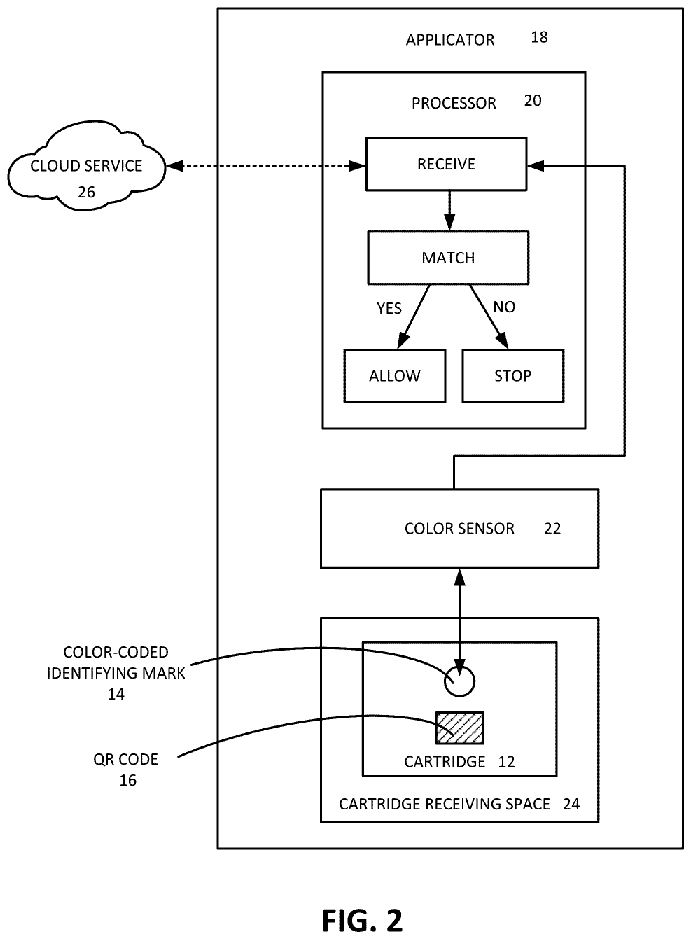

As shown in FIG. 2, the applicator can identify the cartridge (based on the associated color code) and contact the cloud service 26 for information about the identified cartridge. The applicator 18 can also detect a presence or an absence of a cartridge in a cartridge receiving space 24. A selected cartridge 12 can be positioned within the cartridge receiving space 24 of the applicator 18. The applicator 18 can detect the color code (color-coded identifying mark 14) of the cartridge 12 so that the authentication can begin. It is assumed that the color code has already been previously linked to the applicator 18 by the scanning of the QR code 16 with mobile device (not illustrated in FIG. 2) and uploading the associated data and the applicator 18 to a database on the cloud service 28. Thus, when the applicator 18 scans the color code of the cartridge 12, at least a part of the associated data related to the cartridge 12 can be retrieved from the cloud service 28.

The applicator 18 can include at least a color sensor 22, which can be shielded from the effects of outside light, and a processor 20. The color sensor 22 can detect the color of the color-coded identifying mark 14 on the cartridge 12 and can send the color to the processor 20. For example, the color sensor 22 can identify one or more pixels of the color-coded identifying mark 14, affording the color sensor 22 and/or the processor 20 the ability to distinguish between different types of medication being taken at the same time by a patient. As an example, the color sensor 22 can have three different hue channels (red, green, blue (RGB)), so color coding can have three channels with five levels of color saturation or gray scale, and printed colors can thus afford 5³ levels, or 125 different values, nearly equivalent to 7 bits of information. Such information has been confirmed through extensive testing of color sensors. In practice, patients are unlikely to use more than two or three medications at once, so there is more than enough color encoded information to distinguish different types. It should be noted that printed color codes can be stable in hue and color saturation over an extended time (e.g., 6 months, one year, two years, three years, or the like) for this encoding scheme to be fault tolerant. For example, printed medical grade labels with a top transparent waterproof coating can be marring and UV fade resistant, which are commonly available, can by utilized for the printed color code. The printed color code can therefore remain stable for longer than the expiration date of most drugs, including the drug. For example, the printed color code can remain stable for several (e.g., two or three) years.

The processor 20 can receive information/data about the color of the color-coded identifying mark 14 on the cartridge 12 from the color sensor 22 and can receive profile information from the cloud service 26. The profile information can include stored information associated with the at least one cartridge (e.g., obtained from the data associated with the QR code(s) 16) such as at least color information for the color-coded identifying mark on each of the at least one cartridge. The profile information can also include user specific information (e.g., bibliographic information, demographic information, health information, prescription information, etc.). The processor 20 can match the color of the color coded-identifying mark to the color information from the cloud service 26. If the color is a match with the color information, then the processor 20 can allow the applicator 18 to apply the therapeutic agent from the cartridge 12. If the color does not match with the color information, then the processor 20 can stop the applicator 18 from applying the therapeutic agent from the cartridge 12. The color may not match because the selected cartridge 12 is a counterfeit, is the wrong cartridge for a specific time of day, has already been applied for a maximum dosage of a time period, the cartridge is expired, or the like.

While not shown in FIG. 2, after the processor 20 of applicator 18 has matched and authenticated a cartridge 12 for use the processor 20 can also track information about the cartridge and its therapeutic agent. For example, the processor 20 can track uses and times of the uses of the applicator to apply the therapeutic agent from the cartridge. In another example, the processor 20 can track a volume of the therapeutic agent remaining in a given cartridge based on the number of uses of the cartridge in the applicator. Any portion of this information (up to all of the information) can be transmitted to a user's profile in the database on the cloud server and/or to the mobile device of the user. The age of the cartridge can be tracked in the cloud based upon the first registration date of the QR code. The processor can also determine if the cartridge receiving space of the applicator is empty based on the color sensor detecting a color of the cartridge receiving space. The processor 20 may upload this determination to the cloud server and/or create an alert (audio, visual, or haptic) via the applicator 18 and/or the mobile device that the space is empty.

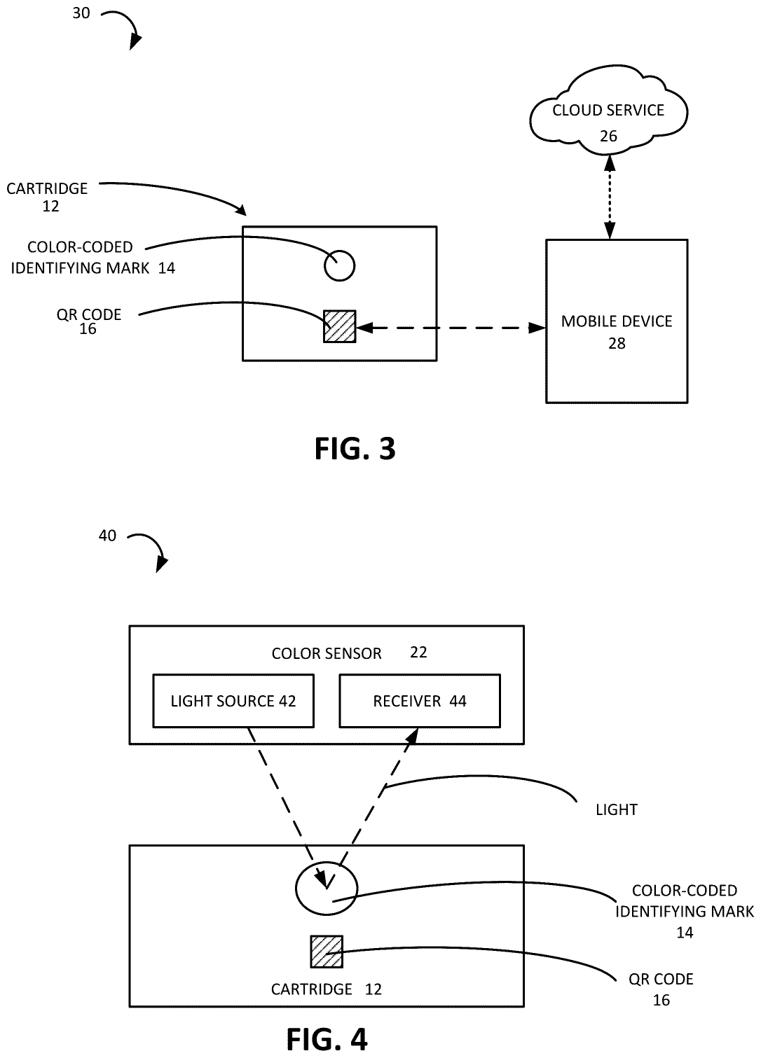

FIG. 3 shows an example of how the cloud service (or databases on a cloud service) are populated for the applicator (not shown) by partial system 30 that can be done with each cartridge a patient obtains. The cartridge can have a color-coded identifying mark 14 (described with regard to FIG. 2) and a QR code 16 on the exterior of the cartridge. The mobile device 28 can be used to scan the QR code 16 (e.g., with a camera, or another image sensor). As mentioned above, the QR code 16 can be associated with data that includes at least the color information of the color-coded identifying mark associated with the therapeutic agent of a cartridge and dosage information related to the therapeutic agent for the user. This information can be associated uniquely with a single user's applicator. In one example, the information can be associated with the applicator and housed in a HIPPA compliant platform so that usage data and compliance are not accessible to outside observers (only the user, any user-appointed associates, and one or more medical professionals).

The mobile device 28 can access the data associated with the QR code 16 and upload at least a portion of the data to the profile information of the user stored in a database on the cloud service. The mobile device 28 may also save the data in local memory. If the QR code 16 is incorrect (e.g., a counterfeit) then no data can be accessed and the mobile device 28 may notify and/or alert the user, and optionally a third party (e.g., a distributor) associated with the non-counterfeited cartridge and/or affected by the counterfeited cartridge. As an example, the notification may include data that the cartridge is a fake and may include a knock off or wrong therapeutic agent (which may be harmful or not effective).

FIG. 4 shows a more detailed view 40 of an example of the color sensor 22 of applicator 18. The color sensor 22 can detect the color code without physically contacting the color-coded identifying mark 14. The color sensor 22 can include a light source 42 that can emit a constant homogenous light. The light source 42 can be, for example, a white light emitting diode (LED). Having an internally controlled bright LED light source is located inside the housing to give a stable background lighting as external background lighting warmth and tint can throw off the measured value of the color code. The light source 42 can be positioned to illuminate the color-coded identifying mark 14 when the cartridge is in the cartridge receiving space (not shown in FIG. 4). The light source may be pulsed on and off to enable background measurements of color to be subtracted out to improve color measurement accuracy. The illumination may be a directed light beam with focusing micro optics. The color sensor 22 can also include a receiver photosensor 44 that can detect the color of the illuminated color-coded identifying mark 14 of the cartridge. The receiver 44 can be at least one of a photodiode, a photodetector, a photomultiplier tube, or the like that can detect color from a single pixel. The color sensor 22 can identify at least 125 different color variations, for example. As an example, the color sensor 22 can be easily calibrated and verified in manufacturing by using test cartridges of different hues and saturations. The color sensor 22 can be shielded from light external to the applicator (not shown). As an example, the cartridge can be placed in the cartridge receiving space so that the color-coded identifying mark 14 faces the back and the rest of the cartridge acts as a shield; the light source 42 and the receiver 44 of the color sensor 22 can be operational to detect the color while external light is blocked by the cartridge. As another example, an outer housing or body of the apparatus can block or shield the area where the light source 42 and the receiver 44 of the color sensor 22 can operate.

### IV. Methods

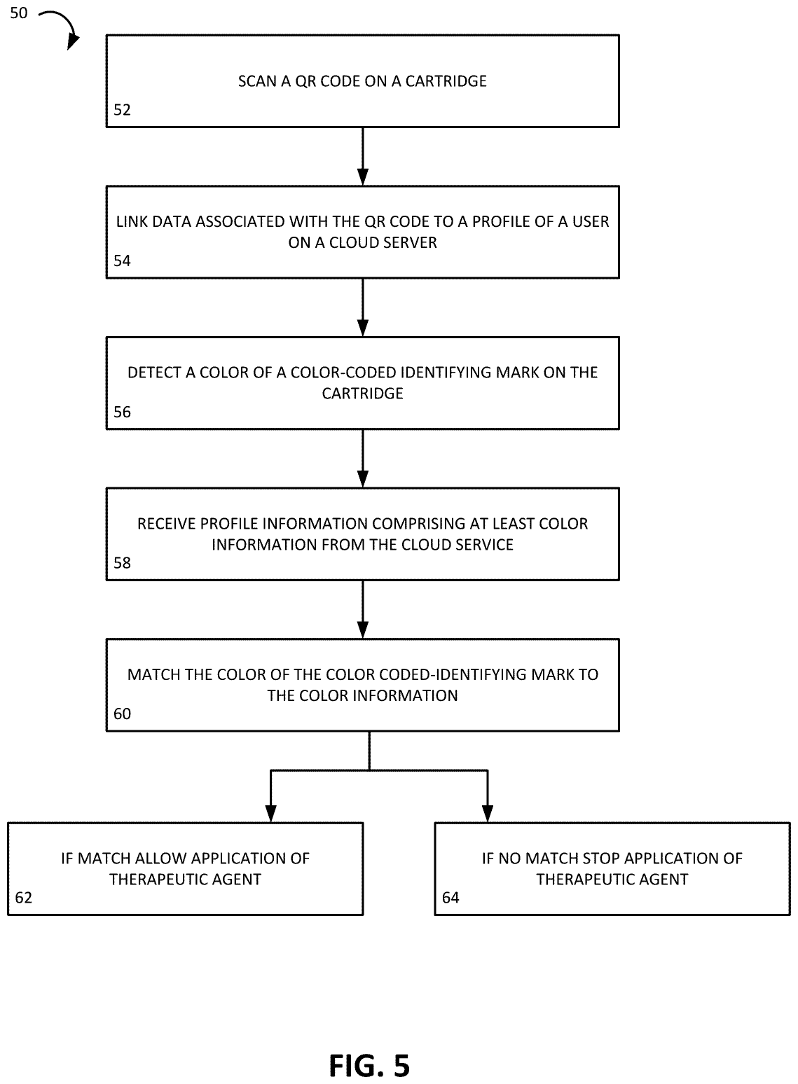

Another aspect of the present disclosure can include method 50 (FIG. 5) for authenticating a cartridge holding a therapeutic agent using data linked to an apparatus to apply the cartridge stored in a cloud service. The method 50 is described as working with a single pixel, but it should be understood that the single pixel is merely an example that can be extended to two or more pixels such that the number of machine readable combinations can be extended by a factor of 2^{N}, where N is a positive integer. The method 50 can be executed with the system 10 and the applicator device 18 described above with respect to FIGS. 1-4. The system 10 of FIG. 1 shows an example of various components that can be used to execute the method 50.

The method 50 is illustrated as process flow diagram with flowchart illustrations. For purposes of simplicity, the method 50 is shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the method 50.

Referring now to FIG. 5, illustrated is a method 50 for authenticating a cartridge holding a therapeutic agent for delivery via an applicator (e.g., applicator 18). The method 50 can include steps 52-54 that include uploading information regarding a QR code scanned by a mobile device to a cloud service (e.g., one or more associated databases) and associating the code with a certain applicator. The method 50 can also include steps 56-60, which include scanning a color code of a cartridge by an applicator and determining whether the cartridge should be used by the applicator.

At step 52, a QR code (or other type of bar-code) on an exterior of a cartridge storing a therapeutic agent can be scanned (e.g., by a system comprising at least a processor and an imaging component, such as mobile device 28). The QR code or other type of bar-code can be associated with data related to at least color information of a color-coded identifying mark associated with the therapeutic agent and dosage information related to the therapeutic agent for a user of the therapeutic agent and the system. At step 54, the data associated with the QR code or bar-code can be linked (or uploaded) to a profile of the user stored in a database on a cloud service. The data associated with the QR code or bar-code can become profile information for the user. Steps 52 and 54 can occur at any time before the rest of the steps of method 50 and can represent the first step in a two-step authentication process. Additionally, the data can be linked to an applicator that will be used to deliver the therapeutic agent to a user.

At step 56, a color of a color-coded identifying mark on a cartridge positioned in an applicator of the system (e.g., applicator 18) can be detected via a color sensor of the system (e.g., color sensor 22). As an example, the color sensor 22 can be easily calibrated and verified in manufacturing by using test cartridges of different hues and saturations. The detected color can be sent to, and received by, a processor of the system (e.g., processor 20). At step 58, profile information can be received from the cloud service. The profile information can include stored profile information associated with the at least one cartridge and the stored profile information can include at least color information of the color-coded identifying marks of each of the at least one cartridge from which the cartridge was selected (e.g., the group of cartridges associated with the user via the first step of the authentication process). At step 60, the color of the color-coded identifying mark can be matched with the color information from the stored profile information. The processor can know what color it should be matching for based on, for example, prior instructions, time of day, and/or previous applications of the same or other therapeutic agents within a time period. At step 62, if the color matches, then the applicator can be allowed to apply the therapeutic agent. At step 64, if the color does not match, the applicator can be stopped from applying the therapeutic agent.

Other steps, which are not illustrated, may be a part of method 50. One such step can include tracking uses of the therapeutic applicator to apply the therapeutic agent of the cartridge and time and date information of the uses; and tracking a volume of the therapeutic agent remaining in the cartridge based on the uses. By linking the color-code to the QR code or bar-code and applicator code verified in the cloud upon initial usage, the age of the cartridge can be tracked for a single user. As another example, it can be determined which of two cartridges each having the same therapeutic agent is newer and which is older by matching differences in color codes to differences in manufacturing expiration dates, ordering dates, and/or actual user usage dates of each of the two cartridges—this ensures that a user does not accidentally keep an expired cartridge. The color codes can be unique for a time period (e.g., 5-10 years) with color codes not being reused for the time period in which they are unique. In another example, the absence of a cartridge in the applicator can be determined by the color sensor detecting a color of the cartridge receiving space, where the color of the cartridge receiving space is only detectable in the absence of a cartridge in the cartridge receiving space. A notification (audio, visual, and/or haptic) can be sent to the user that no therapeutic agent can be applied because the applicator is empty.

### V. Examples of Use in Practice

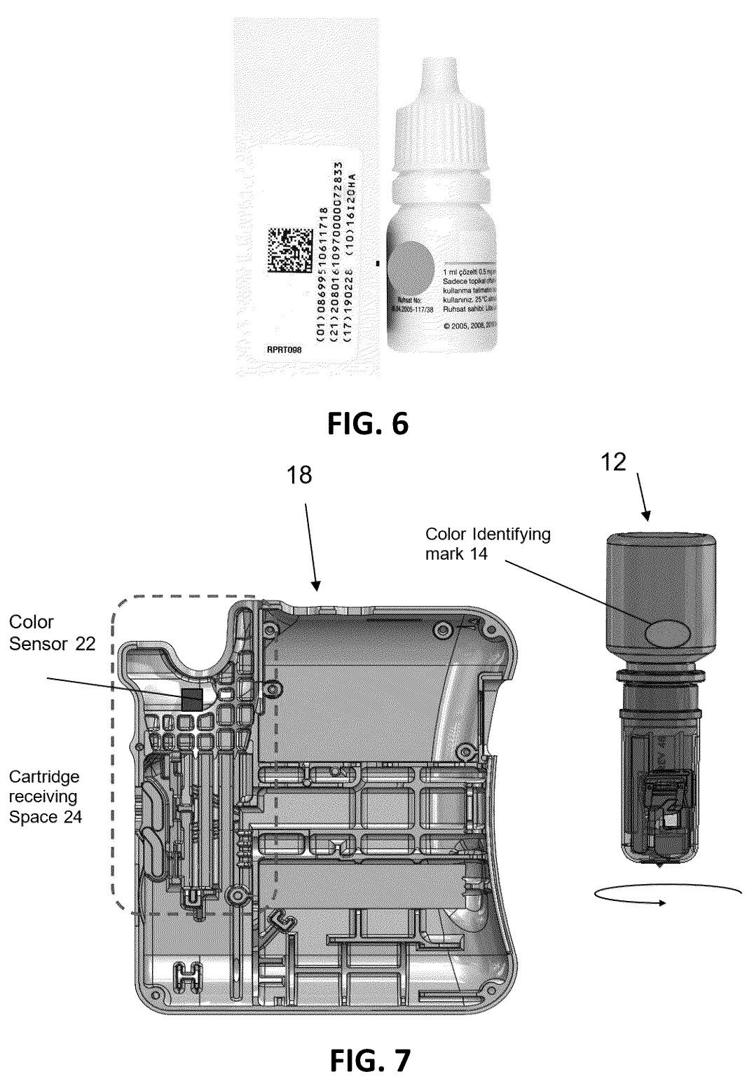

FIGS. 6 and 7 show example real-world embodiments of the systems and devices described herein. FIG. 6 shows an example of a high resolution QR code (e.g., QR code 16 of FIG. 1) printed on packaging and a color code (e.g., color-coded identifying mark 14 of FIG. 1) printed on a bottle for ophthalmic therapeutic agent delivery (e.g., the cartridge(s) 12 of FIG. 1). FIG. 7 shows an example implementation of a cartridge with a color identifying mark (right side of drawing) and an applicator (left side of drawing). The cartridge and applicator of FIG. 7 can be part of the system 10 of FIG. 1—with applicator 18 including the color sensor 22 within the cartridge receiving space 24 and a cartridge 12 with color-coded identifying mark 14). The system shows how the cartridge 12 can fit in the cartridge receiving space 24 with the color-coded identifying mark 14 facing and at least partially lining up with the color sensor 22.

From the above description, those skilled in the art will perceive improvements, changes, and modifications. Such improvements, changes and modifications are within the skill of one in the art and are intended to be covered by the appended claims.

The following is claimed:

1. A system comprising:
at least one cartridge having a color-coded identifying mark on an exterior of the at least one cartridge, wherein the at least one cartridge is configured to hold a therapeutic agent and the color-coded identifying mark is unique to the therapeutic agent; and
an applicator configured to deliver the therapeutic agent from the cartridge to a user, the applicator comprising:
a cartridge receiving space configured to hold a cartridge, wherein the cartridge is selected from the at least one cartridge,
a color sensor configured to detect the color of the color-coded identifying mark on the cartridge, and
a processor in communication with a cloud service and configured to:
receive the color of the color-coded identifying mark on the cartridge from the color sensor;
receive profile information from the cloud service, wherein the profile information comprises stored information associated with the at least one cartridge, wherein the stored information comprises at least color information of the color-coded identifying marks of the at least one cartridge; and
match the color of the color-coded identifying mark to the color information, wherein:
if the color matches, allow the applicator to apply the therapeutic agent, and
if the color does not match, stop the applicator from applying the therapeutic agent.

2. The system of claim 1, wherein the at least one cartridge has a QR code or other bar-code printed on an exterior of the at least one cartridge or its associated packaging that is associated with data, wherein the data comprises at least the color information of the color-coded identifying mark associated with the therapeutic agent of the at least one cartridge and dosage information related to the therapeutic agent for the user.

3. The system of claim 2, wherein the system further comprises a mobile device in communication with the cloud service, wherein the mobile device is configured to scan the QR code or the other bar-code of the at least one cartridge, access the data associated with the QR code or the other bar-code, and upload at least a portion of the data to the profile information of the user on the cloud service.

4. The system of claim 3, wherein the processor is further configured to:
track uses and times of the uses of the applicator to apply the therapeutic agent from the cartridge; and
track a volume of the therapeutic agent remaining in the cartridge based on uses of the cartridge in the applicator.

5. The system of claim 1, wherein the processor is further configured to determine which of two cartridges each having the same therapeutic agent is newer and which is older by matching differences in color codes to differences in manufacturing expiration dates, ordering dates, and/or actual user usage dates of each of the two cartridges.

6. The system of claim 1, wherein the processor is further configured to determine whether the cartridge receiving space is empty based on the color sensor detecting a color of the cartridge receiving space.

7. The system of claim 1, wherein the color sensor comprises:
a light source emitting a constant homogenous light and positioned to illuminate the color-coded identifying mark when the cartridge is in the cartridge receiving space; and
a receiver configured to detect the color of the illuminated color-coded identifying mark.

8. The system of claim 7, wherein the light source is a white LED.

9. The system of claim 1, wherein the color sensor is configured to identify at least 125 different color variations.

10. The system of claim 1, wherein the color sensor is configured to detect color in a single pixel.

11. The system of claim 1, wherein the cartridge receiving space is shielded from external lighting.

12. An applicator configured to deliver a therapeutic agent to a user, the applicator comprising:
a cartridge receiving space configured to hold a cartridge having a color-coded identifying mark on an exterior of the cartridge, wherein the cartridge is configured to store the therapeutic agent delivered by the applicator;
a color sensor configured to detect the color of the color-coded identifying mark on the exterior of the cartridge, wherein the color-coded identifying mark is unique for the therapeutic agent; and
a processor in communication with a cloud service and configured to:
receive the color of the color-coded identifying mark on the cartridge from the color sensor;
receive profile information from the cloud service, wherein the profile information comprises stored information associated with the cartridge, wherein the stored information comprises at least color information of the color-coded identifying marks of at least one cartridge associated with the user; and
match the color of the color-coded identifying mark to the color information, wherein:
if the color matches, allow the applicator to apply the therapeutic agent, and
if the color does not match, stop the applicator from applying the therapeutic agent.

13. The applicator of claim 12, wherein the color sensor comprises:
a light source emitting a constant homogenous light and positioned to illuminate the color-coded identifying mark when the cartridge is in the cartridge receiving space; and
a receiver configured to detect the color of the illuminated color-coded identifying mark.

14. The system of claim 13, wherein the light source is a white LED.

15. The system of claim 12, wherein the color sensor is configured to detect color in a single pixel of the color-coded identifying mark on the cartridge.

16. The system of claim 12, wherein the cartridge receiving space further comprises an external light shield configured to block all external light.

17. A method for authenticating a cartridge holding a therapeutic agent, the method comprising:
scanning, via a system comprising a processor, a QR code or a bar-code on an exterior of a cartridge or packaging associated with the cartridge during purchase storing a therapeutic agent, wherein the QR code or the bar-code is associated with data related to at least color information of a color-coded identifying mark associated with the therapeutic agent and dosage information related to the therapeutic agent for a user of the therapeutic agent and the system;
linking, via the system, the data with a profile of the user on a cloud service;
detecting, via a color-sensor of the system, a color of a color-coded identifying mark on the cartridge;
receiving, by the system, profile information from the cloud service, wherein the profile information comprises stored information associated with the at least one cartridge, wherein the stored information comprises at least color information of the color-coded identifying marks of the at least one cartridge; and
matching, by the system, the color of the color-coded identifying mark to the color information, wherein:
if the color matches, allow the applicator to apply the therapeutic agent, and
if the color does not match, stop the applicator from applying the therapeutic agent.

18. The method of claim 17, further comprising:
tracking, by the system, uses of the therapeutic applicator to apply the therapeutic agent of the cartridge and time and date information of the uses; and
tracking, by the system a volume of the therapeutic agent remaining in the cartridge based on the uses.

19. The method of claim 17, further comprising determining, by the system, which of two cartridges each having the therapeutic agent is newer and which is older by matching differences in color codes to differences in manufacturing expiration dates, ordering dates, and/or actual user usage dates of each of the two cartridges.

20. The method of claim 17, further comprising determining the absence of the cartridge based on the color sensor detecting a color of a cartridge receiving space of the system, wherein the color of the cartridge receiving space is only detectable in the absence of the cartridge in the cartridge receiving space.
